(19) United States
(12) Patent Application Publication
(10) Pub. No.: US 2006/0259000 A1
(43) Pub. Date: Nov. 16, 2006

(54) Protection disposable under drawers for men with a opening in the front

(75) Inventors: Evelyn Bailey, Blueisland, IL (US); Keisha Wiggins, Gary, IN (US); Gay Johnson, Gary, IN (US); Shelia Johnson, Waukegan, IL (US)

Correspondence Address: EVELYN BAILEY, 12202 Gregory St., Blue Island, IL, 60406, US

(73) Assignee: Evelyn Bailey

(21) Appl. No.: 11/093,072

(22) Filed: May 16, 2005

(51) Int. Cl. A61F 13/15

(52) U.S. Cl. 604/385.09

(57) ABSTRACT

For Men with an Snap Opening in the Front Take this invention as one which will allow men to be able to use the restroom with less stress and without having to embarrassingly undress themselves to urinate. This can be resolved by sewing an opening with a snap in front of disposable under-drawers. There is a handicap for men that need to wear a protective undergarment without having an opening in the front to urinate through. My invention will be beneficial to many men.

My invention is one in which will finally allow men to be able to use the restroom with less stress and without having to embarassingly undress themselves just to urinate-by sewing an opening in the front of the Disposable Under Drawers . I think it is a handicap for men that need to wear a protective undergarment without having a opening in the front to urinate through. There may be some but I have not found any in the four years that I have been caretaker of my husband. I have been sewing an opening in the front of the store bought disposable garments for the last few months. Then I had a thought that this sewn opening in the front of the disposable undergarments could be beneficial to so many men.

This is my invention.

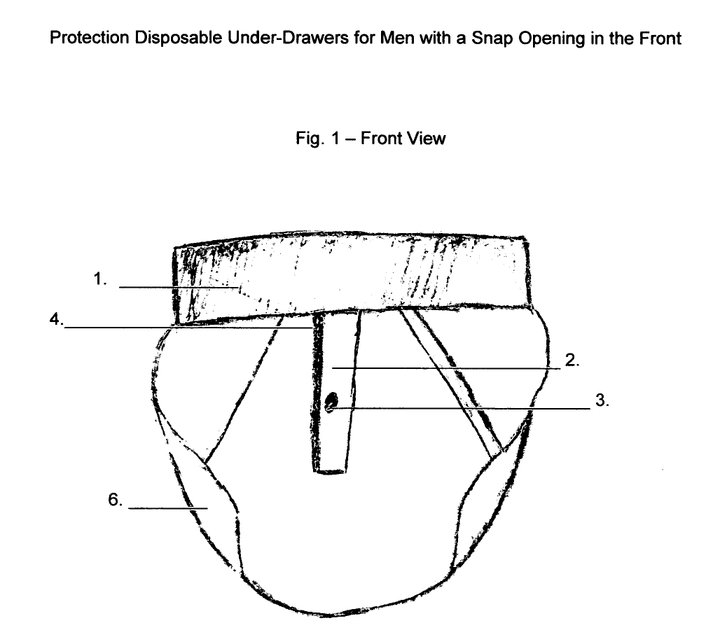

FIG. 1-Front View
1. 1 1/2″ elastic leak barrier waistband.
2. 1 1/2″ left side waterproof leak guard with snap.
3. Plastic snap.
4. 8″ snap opening to allow easier urination.
6. Interior leg leak guard to protect from any accidents that may occur.

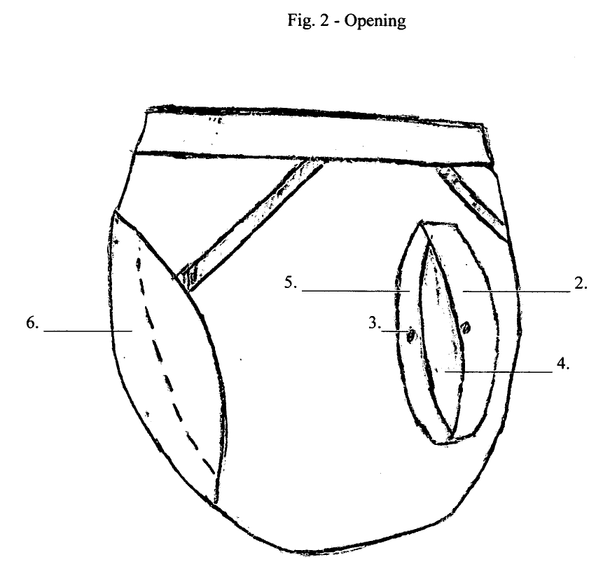

FIG. 2-Opening
3. Plastic Snap
4. 8″ snap opening to allow easier urination.
2. 1 1/2″ left side waterproof leak guard with snap.
5. 1″ right side waterproof leak guard with snap.
6. Interior leg leak guard to protect from any accidents that may occur.

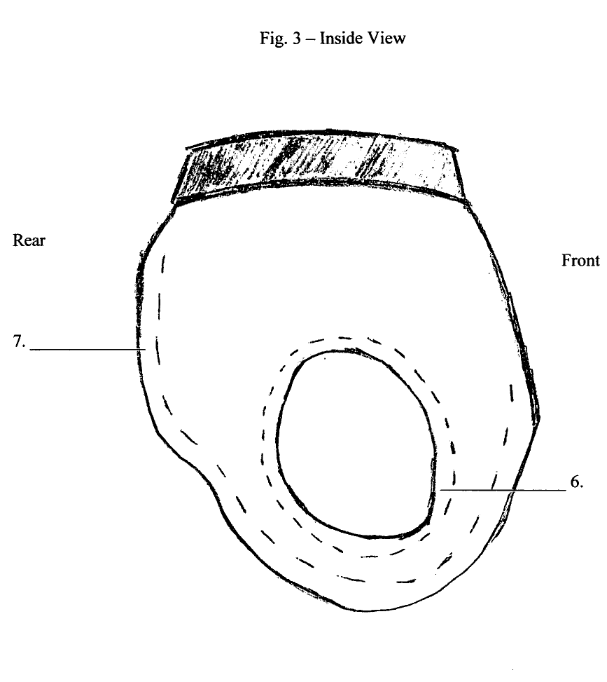

FIG. 3-Inside View
6. Interior leg leak guard for protection from any accidents that may occur.
7. Inside front and rear leak guard for extra protection.

### BRIEF SUMMARY OF THE INVENTION

My invention allows for men that need incontinence care but are not totally unable to go to the washroom to urinate on their own. With this design these disposable under-drawers have extra security leak-proof lining in the front, rear, and around the legs of the under-drawers. What makes this different is that they fit comfortably like regular men underwear with an easy to open and close 1 1/2 inch waterproof snap front. On the drawings you will see that the opening is 8 inches long with a plastic snap in the middle, which will make the front panel of the under-drawers very easy for men to function as they would normally. Times when they cannot control their urination and bowel movements they will still have security.

The Protection Disposable Under-Drawers for Men with a Snap Opening in the Front will alleviate the stressful and embarrassing moments of having to undress themselves to urinate. There may be some, but I have not found any in the four years that I have been caretaker of my husband. I have been sewing an opening in the front of store bought disposable garments for the last few months. Then I had a thought that this sewn opening in the front of the disposable undergarments would be beneficial to so many men.

This is my invention.

1. Take my invention as the Protection disposable under-drawers for men, with a snap opening in the front.
